(19) United States
(12) Patent Application Publication
(10) Pub. No.: US 2002/0156427 A1
(43) Pub. Date: Oct. 24, 2002

(54) Needle protector assemblage, syringe equipped therewith and needle hub

(76) Inventors: Hitoshi Suzuki, Yokohama-shi (JP); Seiichi Takigawa, Yokohama-shi (JP)

Correspondence Address: KUBOVCIK & KUBOVCIK, SUITE 710, 900 17TH STREET NW, WASHINGTON, DC, 20006

(21) Appl. No.: 10/126,613

(22) Filed: Apr. 22, 2002

(30) Foreign Application Priority Data
Apr. 24, 2001 (JP) 2001-125233
Jul. 26, 2001 (JP) 2001-225651

(51) Int. Cl. A61M 5/32

(52) U.S. Cl. 604/198

(57) ABSTRACT

The invention provides a needle protector assemblage which contributes to ensuring the safety of a sequence of operations of sucking up fluid through a needle, protecting the needle through which the fluid has been drawn, injecting the fluid into a patient, and housing the needle after injection and also provides the needle protector assemblage including a needle hub (11) of a needle (10), a protector (32) capable of covering the needle (10), and a fixing member (31) which is formed as a continuity of the protector (32) and fixes the needle protector assemblage on the outer perimeter of the needle hub (11) of the needle (10). In this needle protector assemblage, the needle hub (11) includes an outer-perimeter-engaging portion (11b) capable of engaging with the fixing member (31) and an idling portion (11d) capable of idling the fixing member (31).

### BACKGROUND OF THE INVENTION

1. Field of the Invention

The present invention relates to a needle covering member for covering the points of used needles, including used indwelling needles, and an injection device equipped therewith.

2. Description of the Related Art

The prior arts will be described with reference to FIGS. 8, 9 and 10.

When using an injector for a patient, the needle must be stuck with its beveled tip portion (hereinafter referred to as “tip surface”) facing up. Before that, the needle hub and the syringe must be turned relatively to each other.

As shown in FIG. 8, when drawing fluid in a syringe, the tip surface must be placed in a predetermined direction relative to a cap of a bottle containing the fluid. Specifically, as shown in FIGS. 8(a) or (b), the needle must be stuck to the cap in a way shown in FIG. 8(c) or (d). This is because the tip of the needle scrapes off the cap if it is stuck in a way shown in FIG. 8(e). On the other hand, since a person must draw the fluid while reading the scale marked on the barrel portion of a syringe, it is necessary for him or her to turn the needle hub and the syringe relatively to each other.

Moreover, a used needle has a risk of exposing people to, for example, AIDS or hepatitis infection. In order to avoid the risk, there have been provided various types of injectors equipped with protectors which protect people from accidental contact to the point the used needle.

An injector equipped with a protector is used as follows. First, a cover cap covering the needle tip portion of the injector is dismounted, and then fluid is drawn through the exposed needle tip. After the fluid is injected into a patient, the protector is moved to an adequate position and the whole injector is discarded.

An injector equipped with a protector will be described as follows with reference to FIG. 9. In a conventional needle with no protector, as shown in FIG. 9(a), an inner perimeter of a needle hub 11 of the needle 10 is fitted to an outer perimeter of a tip portion 22 of the syringe 20, which has been tapered by 6%, so that both tapered surface may fit to each other. At that time, the relative mounting angle between the tip surface 10a of the needle and the syringe 20 is not uniform.

Generally, when fixing a protector to a syringe 20 and a needle 10, the protector 30 is positioned between the needle 10 and the syringe 20, as shown in FIGS. 9(b) and (c). Specifically, an inner perimeter of a fixing member 31 of the protector 30 is fitted to an outer perimeter of a tip portion 22 of the syringe 20, and an inner perimeter of the needle hub 11 is fitted to an outer perimeter at the tip side of the fixing member 31. This procedure is, for example, adopted in a prior art disclosed in JP 6-315532A.

This setting procedure, however, has a problem that a dead space in which the fluid accumulates enlarges and that the fluid cannot be pushed out even by pushing the plunger to the syringe 20. The setting procedure that solves this problem is shown in FIG. 10. Specifically, an inner perimeter of a needle hub 11 is fitted to an outer perimeter of a tip portion 22 of a syringe 20, and an inner perimeter of a fixing member 31 of a protector 30 is fitted to an outer perimeter at the tip side of the needle hub 11. This procedure is, for example, adopted in a prior art disclosed in JP 10-80486A. According to the procedure, a dead space due to a use of a protector will not enlarge.

Since relative position between a needle hub and a syringe differs depending on doctors or the like who give an injection to a patient, it is inevitable for them, when they use the injectors, to turn the needle hub and the syringe relatively to each other. However, when using an injector equipped with a protector, as shown in FIG. 10, since the fixing member 31 of the protector 30 is positioned on the outer perimeter of the needle hub 11, there has occurred a problem that it is difficult to turn the needle hub 11 and the syringe 20 relatively to each other. In other words, there has occurred a situation that only the fixing member 31 of the protector 30 is turned even though the needle hub 11 is intended to turn relatively to the syringe 20, or that the needle hub 11 slips off from the tip portion 22 of the syringe 20 when the needle hub 11 is turned together with the fixing member 31.

Further, after the needle hub and the syringe are turned relatively to each other, a person draws fluid through a needle while reading the scale marked on the barrel portion of a syringe. At that time, if the protector 30 is not fitted adequately, there may occur a problem that the protector hides the scale or that the needle tip cannot be seen. Accordingly, it is necessary to turn the protector to an adequate position after turning the needle hub and the syringe relatively to each other. Also in this case, however, there has occurred a problem that only the fixing member 31 is turned and that the needle hub 11 slips off from the tip portion 22.

### SUMMARY OF THE INVENTION

The problem to be solved by the present invention is how to provide a technique which ensures, when using a protector, the stability in turning the needle hub and the syringe relatively to each other and in turning the protector and the syringe relative to each other.

Accordingly, the object of the present invention is to provide a needle protector assemblage which ensures the safety in a sequence of operations containing drawing the fluid, protecting the needle after drawing the fluid, injecting the fluid into a patient and housing the used needle.

Another object of the present invention is to provide a syringe which ensures the safety in a sequence of operations containing drawing the fluid, protecting the needle after drawing the fluid, injecting the fluid into a patient and housing the used needle.

Still another object of the invention is to provide a needle hub of a needle which ensures the safety in a sequence of operations containing drawing the fluid, protecting the needle after drawing the fluid, injecting the fluid into a patient and housing the used needle.

In order to accomplish the above objects the present invention provides the means described below.

The present invention relates to a needle protector assemblage comprising a needle hub (11) of a needle (10), a protector (32) capable of covering the needle (10) and a fixing member (31) which is formed as a continuity of the protector (32) and which is fixed to the outer perimeter of the needle hub (11) of the needle (10).

In this needle protector assemblage, the needle hub (11) comprises an outer-perimeter-engaging portion (11b) capable of engaging by friction with the fixing member (31) and an idling portion (11d) capable of idling the fixing member (31).

The phrase “formed as a continuity” in the above description “a fixing member (31) which is formed as a continuity of the protector (32) and which is fixed to the outer perimeter of the needle hub (11) of the needle (10)” includes a function of changing a state, in which the needle (10) is exposed to be capable of stinging, to another state, in which the needle (10) is covered with the protector (32).

The term “engaging by friction” means an engaging state in which the fixing member (31) does not idle along the circumference direction relative to the needle hub (11). The example of such engaging is that a projection formed on the needle hub (11) engages with a hollow formed on the fixing member (31).

The term “idling” means the state in which the fixing member (31) continuously slips along the circumference of the needle hub (11), and even if the fixing member (31) turns relatively to the needle hub (11) in the circumference direction, the needle hub (11) will not turn in the same direction.

The fixing member (31) may be arc-shaped as well as ring-shaped. The arc-shaped fixing member provides an advantage over the ring-shaped one that the degree of freedom in assembly increases.

Thus, when the needle hub (11) of the needle (10) is fixed to the tip of the syringe and the fixing member (31) is fixed to the outer perimeter of the needle hub (11) of the needle (10) by friction engagement, the injector becomes a highly safe injector equipped with a needle protector assemblage.

Moreover, when the tip surface of the needle (10) is turned in use of the injector, the needle hub (11) turns synchronizing with the turning motion of the fixing member (31) which engages by friction with the needle hub (11). Thereby the angle of the tip surface of the needle (10) can be easily adjusted. Moreover, when the fixing member (31) is positioned at the idling portion (11d), the protector (32) alone can be turned. Thereby the position of the protector (32) can be easily adjusted.

Furthermore, the present invention may have a further specific mode of the needle protector assemblages obtained by adding limitation to the above described one, and it may be the needle protector assemblage, wherein one of the outer-perimeter-engaging portion (11b) and the idling portion (11d) of the needle hub (11) is formed at the near side to the needle (10) and the other at the far side from the needle.

In this way, when the outer-perimeter-engaging portion (11b) of the needle hub (11) is formed at the far side from the needle (10) and the idling portion (11d) of the needle hub (11) at the near side to the needle, in case of turning the needle hub (11) relatively to the syringe (20), the fixing member (31) is pushed toward the far side from the needle, i.e., toward the near side to a user. This pushing force acts in such a direction as to fix the needle hub (11) relative to the syringe (20), thereby the needle protector assemblage provides the advantage of preventing the needle hub (11) from slipping off from the syringe more effectively.

On the other hand, when the outer-perimeter-engaging portion (11b) of the needle hub (11) is formed at the near side to the needle (10) and the idling portion (11d) of the needle hub (11) at the far side from the needle (10), it is possible to combine a stopper portion (11c) and the outer-perimeter-engaging portion (11b) as synchronizing projection or the like, thereby the needle protector assemblage provides the advantage of simplifying the die structure used in molding and reducing the number of the parts.

The phrase “at the near side to the needle” herein used means the direction toward the needle tip and the phrase “at the far side from the needle” the direction toward the root of the needle.

Furthermore, the present invention may have a further specific mode of the needle protector assemblages obtained by adding limitation to the above described one, and it may be the needle protector assemblage, wherein the needle hub (11) further includes a stopper portion (11c) for preventing the fixing member (31) from slipping off from the idling portion (11d) toward the needle (10).

In this needle protector assemblage, since the needle hub (11) includes a stopper portion (11c), the fixing member (31) does not slip off toward the needle (10) even while the angle or the position of the tip surface of the needle (10) or the protector (32) is being adjusted.

Furthermore, the present invention may have a further specific mode of the needle protector assemblages obtained by adding limitation to the above described one, and it may be the needle protector assemblage, wherein the inner perimeter of the fixing member (31) includes an engaging portion which engages with the outer perimeter shape of the outer-perimeter-engaging portion (11b) of the needle hub (10).

In this needle protector assemblage, since the inner perimeter of the fixing member (31) includes an engaging portion such as synchronizing concave portion (31a) which engages with the outer perimeter shape of the outer-perimeter-engaging portion (11b) of the needle hub (10), the angle of the tip surface of the needle (10) can be adjusted only by turning the fixing member (31).

The present invention also relates to a syringe equipped with any one of the above-described needle protector assemblages, comprising a piston which enables the discharge and suction of fluid, a barrel portion for containing the fluid and a tip portion, where the needle protector assemblage is equipped, provided at the tip of the barrel portion.

The syringe according to this invention provides a highly safe injector which is equipped with a needle protector assemblage.

The present invention also relates to a needle hub comprising an outer-perimeter-engaging portion (11b) capable of engaging by friction with a needle protector member (30) which comprises a fixing member (31), which is fixed to the outer perimeter of the needle hub (11) of a needle (10), and an idling portion (11d) capable of idling the fixing member (31).

Furthermore, the present invention may have a further specific mode of the needle protector assemblages obtained by adding limitation to the above described one, and it may be the needle hub, wherein one of the outer-perimeter-engaging portion and the idling portion of the needle hub is formed at the near side to the needle and the other at the far side from the needle.

Furthermore, the present invention may have a further specific mode of the needle protector assemblages obtained by adding limitation to the above described one, and it may be the needle hub which further includes a stopper portion (11c) for preventing the fixing member (31) from slipping off from the idling portion (11d) toward the needle (10).

### BRIEF DESCRIPTION OF THE DRAWINGS

FIG. 1 shows the first embodiment of the present invention in a perspective view.

FIG. 2 shows the first embodiment of the present invention in consecutive cross-sectional views.

FIG. 3 shows the second embodiment of the present invention in a perspective view.

FIG. 4 shows the third embodiment of the present invention in a perspective view.

FIG. 5 shows the fourth embodiment of the present invention in which highly functional requirements are realized in a perspective view.

FIG. 6 shows the fifth embodiment of the present invention in a perspective view.

FIG. 7 shows the fifth embodiment of the present invention in consecutive cross-sectional views.

FIG. 8 illustrates the manner in which fluid is drawn into a syringe.

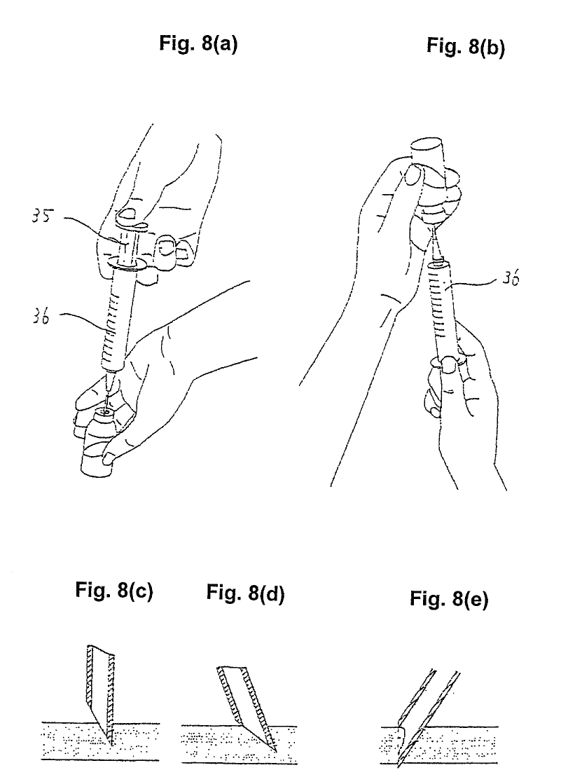

FIG. 9 illustrates a needle hub and its vicinity in a conventional needle.

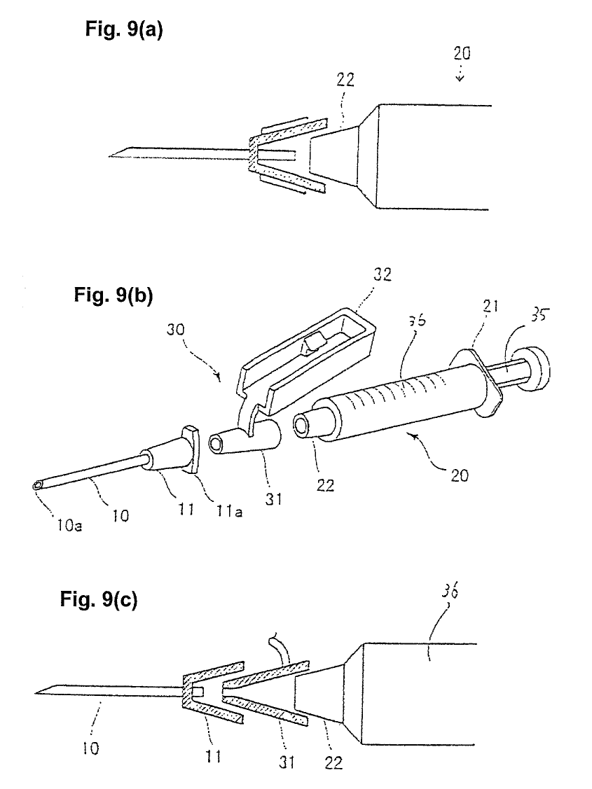

FIG. 10 shows an example of a conventional needle equipped with a safety device in a perspective view.

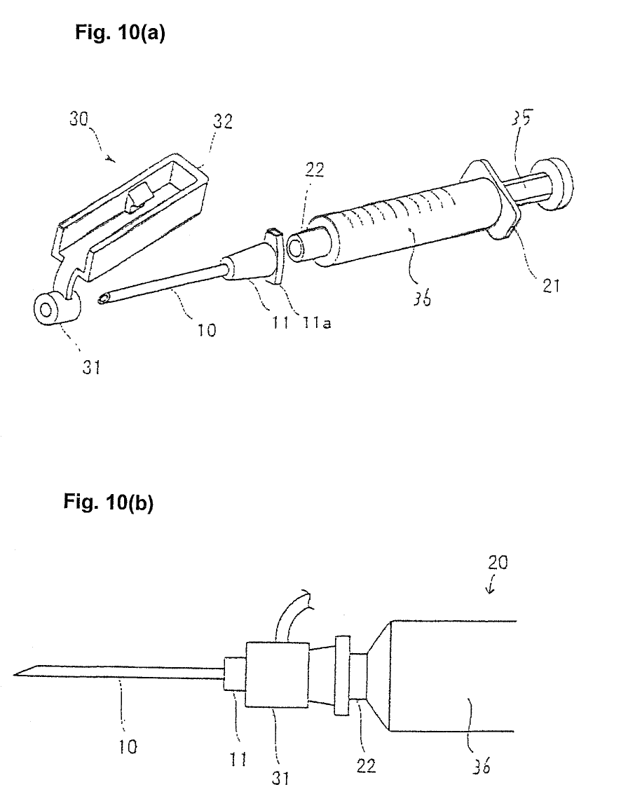

### DETAILED DESCRIPTION OF THE PREFERRED EMBODIMENTS

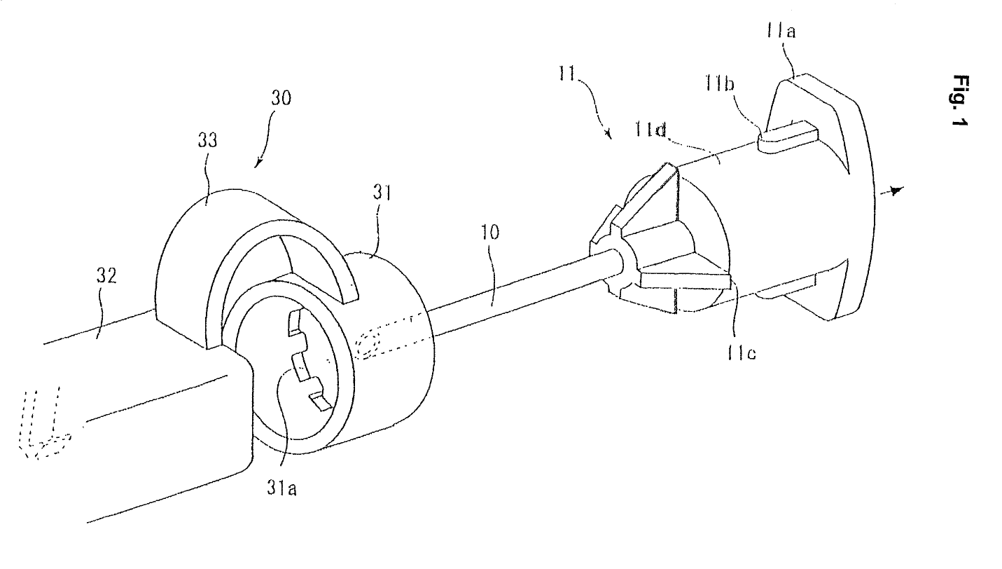

The present invention will be described in further detail in terms of its preferred embodiments with reference to the accompanying drawings as follows. The drawings herein used are FIGS. 1 to 7.

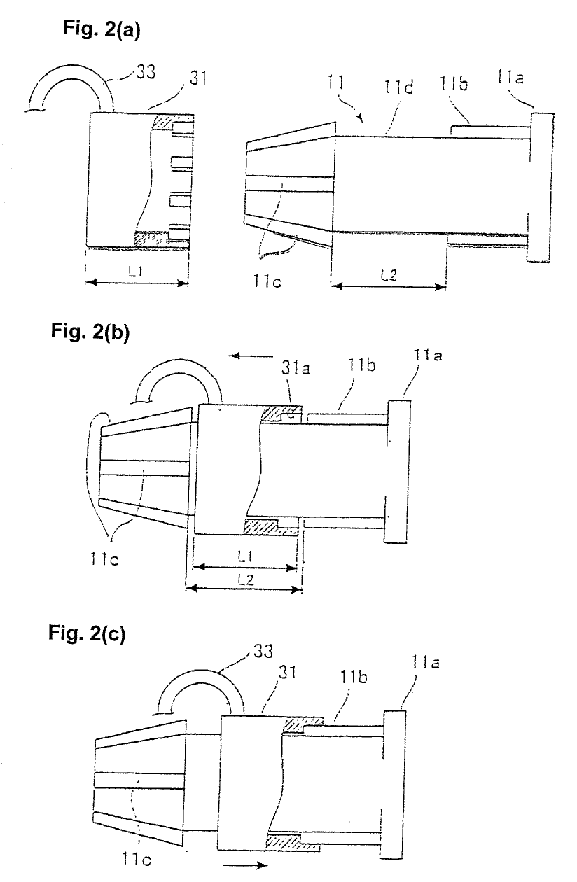

FIGS. 1 and 2 show the first embodiment of the present invention, illustrating the main part of a needle 10 and a needle protector member 30.

The needle 10 is fixed into a needle hub 11. The needle hub 11 comprises a guard portion 11a at an end opposite to the needle 10, a cylindrical body slightly tapered from the guard portion 11a to the needle 10, synchronizing projections 11b projecting outwards, stopper portions 11c projecting from the body portion toward its periphery and an idling portion 11d formed between the synchronizing projection portions 11b and the stopper portions 11c. The synchronizing projection portion (11b) as an outer-perimeter-engaging portion is formed at the far side from the needle and the idling portion (11d) at the near side to the needle.

The needle protector member 30 is a molded form of a thermoplastic resin and comprises a ring-shaped fixing member 31 for fixing to the outer perimeter of the body of the needle hub 11, a box-shaped case body 32 for covering the needle 10 and a bridge-shaped flexible bending portion 33 for connecting the case body 32 and the fixing member 31. At the far side from the case body 32 in the inner perimeter of the fixing member 31 is provided a synchronizing hollow portion 31a engaging with the synchronizing projection portion 11b.

Although the inner perimeter of the fixing member 31 is larger than the outer perimeter of the idling portion 11d of the needle hub 11, once having entered the idling portion 11d, the fixing member 31 does not slip off due to the presence of the stopper portion 11c. At the time of assembly, the fixing member 31 is subjected to elastic deformation from the near side to needle 10 and thereby allowed to climb over the stopper portion 11c.

Supplementary description will be given with reference to FIG. 2. The axial length L1 of the fixing member 31 is shorter than the axial length L2 of the idling portion 11d. Accordingly, the fixing member 31 is allowed to idle smoothly when it is positioned in the idling portion 11d.

The first embodiment has following advantages.

First, the needle hub 11 of the needle 10 is fixed on the tip portion of a syringe, and then on the outer perimeter of the needle hub 11 of the needle 10 the fixing member 31 is fixed by friction engagement. When engaging the fixing member 31 with the needle hub 11, the circular ring of the fixing portion 31 is subjected to slight deformation and thereby allowed to climb over the stopper portions 11c. Thus, there can be provided an injector equipped with the needle protector member and hence of high safety. If the fixing portion 31 and the perimeter of the needle hub 11 should be disengaged, the needle protector member 30 will not be separated from the needle hub 11 immediately.

When turning the tip surface of the needle in use of the injector, by turning the fixing member 31 at the position of the outer-perimeter-engaging portion, the needle hub 11 can be turned synchronizing with the turning motion of the fixing member 31. Thereby the angle of the tip surface can be easily adjusted.

When turning the protector to an adequate position, by turning the fixing member 31 at the position of the idling portion, the protector alone can be turned. Thereby the angle of the protector can be easily adjusted.

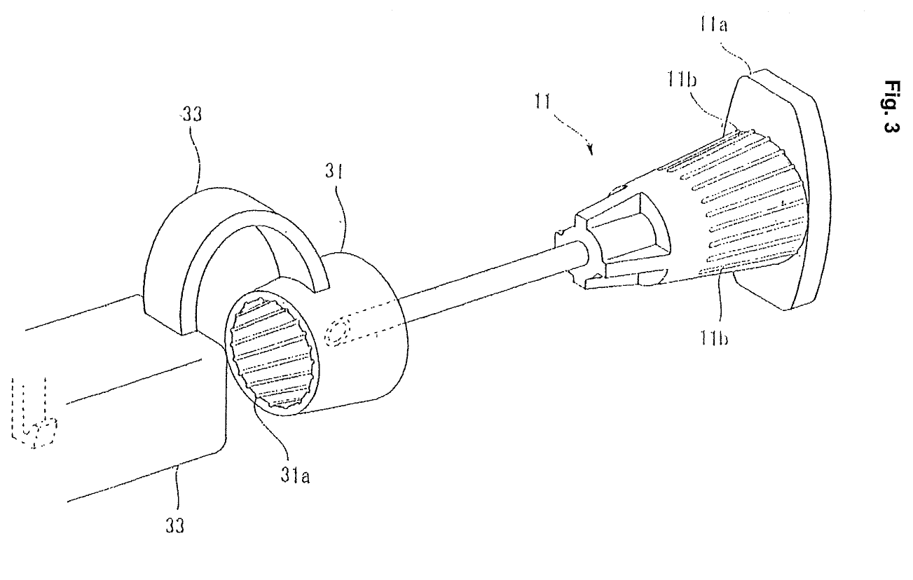

Then the present invention will be described in terms of the second embodiment with reference to FIG. 3. The second embodiment differs from the first embodiment in that a larger number of shorter synchronizing projections 11b as the outer-perimeter-engaging portion formed on the body of the needle hub 11 are provided over the entire perimeter. It goes without saying that the synchronizing hollow portion 31a as an engaging portion provided on the inner perimeter of the fixing member 31 is engaged with the synchronizing projection portion 11b.

Both the outer perimeter of the needle hub 11 and the inner perimeter of the fixing member 31 are tapered axially so that they are easy to assemble.

In order to make simple a cutting die for forming the needle hub 11, the needle hub of this embodiment does not include stopper portions 11c, unlike that of the first embodiment.

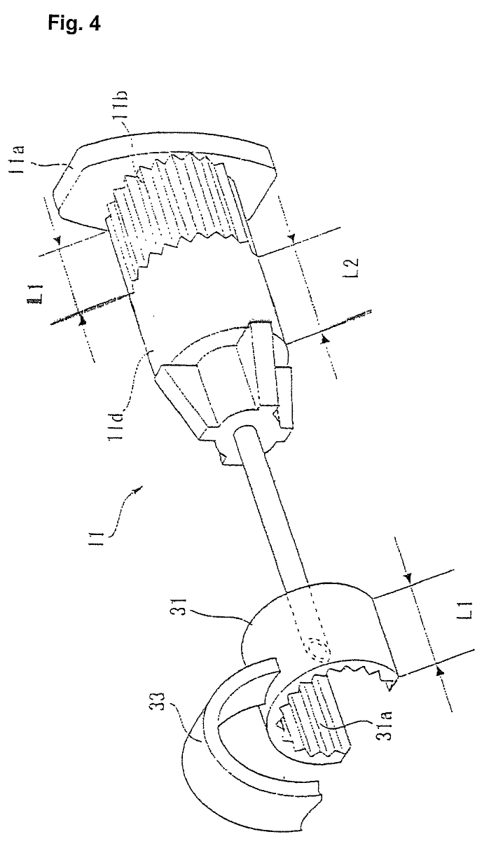

The present invention will be described in terms of the third embodiment with reference to FIG. 4.

This embodiment differs from the second embodiment shown in FIG. 3 in that the fixing member 31 is not ring-shaped but arc-shaped. The use of this fixing member provides the advantage of increasing the degree of freedom in assembly.

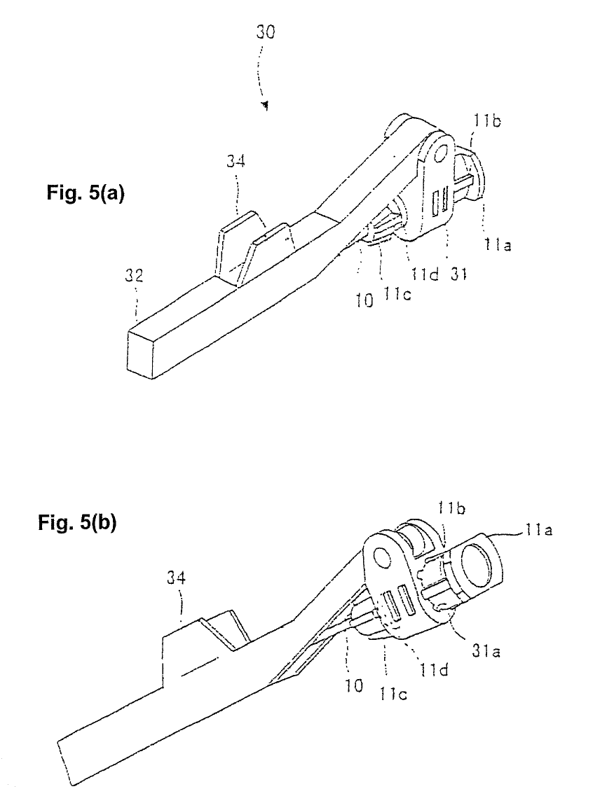

Then the present invention will be described in terms of the fourth embodiment with reference to FIG. 5. The needle protector assemblage of this embodiment is formed in such a manner as to make it possible to select the state in which the needle hub 11 and the fixing member 31 are synchronized with each other when turning and the state in which the fixing member performs an idling motion, and to make it possible to re-expose the needle having been housed in the protector. This needle protector assemblage will be described in further detail.

The needle hub 11 comprises a guard portion 11a formed at its far end from the needle 10, a cylindrical body slightly tapered from the guard portion 11a toward the needle 10, synchronizing projections 11b projecting outward, stopper portions 11c projecting from the body portion toward its periphery and an idling portion 11d formed between the synchronizing projection portion 11b and the stopper portion 11c.

Although the inner perimeter of the fixing member 31 is larger than the outer perimeter of the idling portion 11d of the needle hub 11, once having gone into the idling portion 11d, the fixing member 31 does not slip off due to the presence of the stopper portion 11c. When assembling the protector member 30 and the needle hub 11, the fixing member 31 is subjected to elastic deformation from the side of the needle 10 and thereby allowed to climb over the stopper portion 11c.

First, the needle hub 11 of the needle 10 is fixed on the tip portion of a syringe, and then on the outer perimeter of the needle hub 11 of the needle 10 the fixing member 31 is fixed by friction engagement. When engaging the fixing member 31 with the needle hub 11, the circular ring of the fixing portion 31 is subjected to slight deformation and thereby allowed to climb over the stopper portions 11c. Thus, an injector can be provided which is equipped with the needle protector member and hence of high safety. If the fixing portion 31 and the perimeter of the needle hub 11 should be disengaged, the needle protector member 30 will not be separated from the needle hub 11 immediately.

When turning the tip surface of the needle in use of the injector, by turning the fixing member 31 at the position on the outer-perimeter-engaging portion, the needle hub 11 can be turned synchronizing with the turning motion of the fixing member. Thereby the angle of the tip surface can be easily adjusted.

When turning the protector to an adequate position, by turning the fixing member 31 at the position of the idling portion, the protector alone is allowed to turn. Thereby the angle of the protector can be easily adjusted.

The needle covering member comprises a fixing member 31 as a fixing portion for the needle hub for fixing the needle covering member on the needle hub 11 of the needle 10, a protector 32 for housing and covering the needle 10 and a connecting portion (hinge) for connecting the protector 32 and the fixing member 31 to each other.

The above protector 32 is a box-shaped protector with its underside opened as shown in the perspective views, the opening formed in the underside is a housing opening for getting the needle 10 in and out of the protector 32 and the inner space of the housing opening is a housing space. In the middle of the length of the opposite side surface to the housing opening, provided is a manipulation opening which is in communication with the housing space. To the manipulation opening a pair of manipulation pieces 34 are provided. The pair of manipulation pieces 34 are, though not shown in the figure, hook-shaped pieces with their hooks facing inside, and their ends opposite to the hooks projecting from the housing space, and they are integrated into the protector 32 at the boundary between themselves and the manipulation opening.

The hook-shaped parts described above function as a stop-functioning portion capable of hooking the middle of the length of the needle 10 or its vicinity onto them when the protector 32 is turned relative to the needle 10 toward the housing opening side and covers the needle 10. The protector 32 is designed in such a manner as to allow the spacing between the two facing hook-shaped parts to become smaller than the diameter of the needle 10 when the protector 32 is turned relative to the needle 10 toward the housing opening side.

In the state where the stop-functioning portion described above hooks the middle of the length of the needle 10 or its vicinity, the protector 32 does not turn in such a direction as to expose the needle 10 even under external loads, unless the stop-functioning portion or the needle 10 breaks. Since the stop-functioning portion supports the needle 10 at a position away from the fixing member 31 around which the protector 32 turns, the protector can withstand heavy external loads compared with the structures in which the protector 32 is stopped near the fixing portion 31, in accordance with the principle of the lever. The needle covering member of this embodiment contributes to reducing the consumption of raw materials because it adopts the above described structure.

By the way, though not shown in the figure, the needle covering member of this embodiment includes a needle-stabilizing-functioning portion for stabilizing the needle 10 in the interior of the protector 32 in such a position as to make it easier to release the protector 32 from the stopped state. Although the housing space is large for the thickness of the needle 10, the presence of the needle contacting portion enables positioning of the needle 10 housed in the housing space 32b without offsetting.

The embodiment described above can be provided as an assemblage of an unused needle 10 and a needle covering member, and the assemblage can be packed in such a state that the protector 32 of the needle covering member covers the needle 10 and the stop-functioning portion prevents the needle 10 from being exposed. This makes it unnecessary to protect the needle with a separate member such as cap, leading to reduction of raw materials and wastes accompanying cap manufacture.

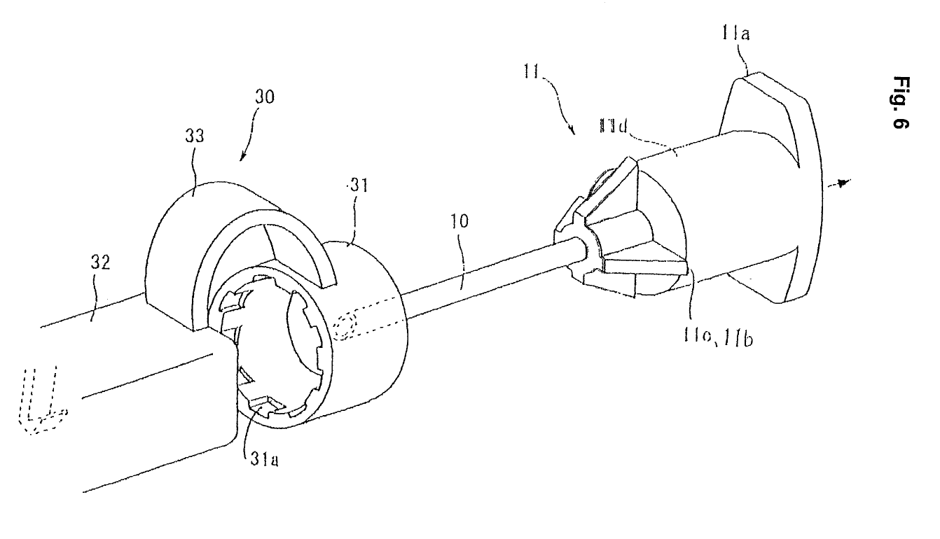

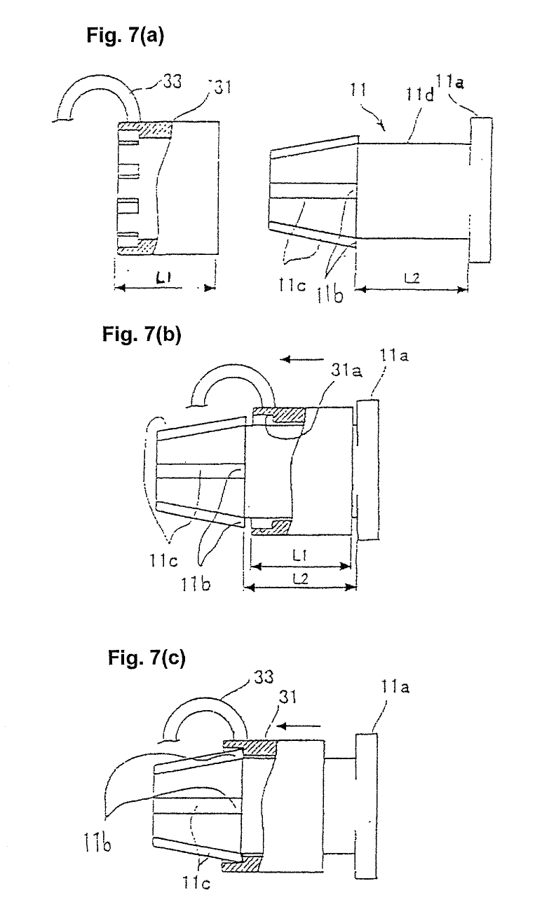

Then the present invention will be described in terms of the fifth embodiment with reference to FIGS. 6 and 7. Since the needle protector assemblage of this embodiment is basically the same as that of the first embodiment shown in FIGS. 1 and 2, the differences between them alone will be described.

The needle protector assemblage of this embodiment differs from that of the first embodiment in that synchronizing projections as an outer-perimeter-engaging portion is formed on the near side to the needle and an idling portion is formed on the far side from the needle and that the synchronizing projections (11b) also serve as stopper portions (11c).

Once having gone into the idling portion 11d, the fixing member 31 does not slip off due to the presence of the stopper portion 11c. When assembling the protector member 30 and the needle hub 11, the fixing member 31 is subjected to elastic deformation from the side of the needle 10 and thereby allowed to climb over the stopper portion 11c.

When turning the tip surface of a needle in use of an injector, a person has only to move the ring-shaped fixing member 31 to the near side to the needle and engage the synchronizing hollows 31a and the synchronizing projections 11b with each other. Thereby the angle of the tip surface can be easily adjusted. When turning the protector 32 alone, a person has only to position the protector 32 at the idling portion 11d. Thereby the angle of the protector can be easily adjusted. Due to the presence of the stopper portions 11c, the needle protector member 30 will not be separated from the needle hub 11 immediately. Furthermore, the needle protector assembly of this embodiment provides advantages of being capable of reducing the number of parts and simplifying the die structure to be used, since the synchronizing projections (11b) serve as stopper portions (11c).

### Industrial Applicability

According to this invention, a needle protector assemblage can be provided which ensures the safety of a sequence of operations of sucking up fluid through a needle, protecting the needle through which the fluid has been drawn, injecting the fluid into a patient, and housing the needle after injection.

Further, according to this invention, a syringe can be provided which ensures the safety of a sequence of operations of sucking up fluid through a needle, protecting the needle through which the fluid has been drawn, injecting the fluid into a patient, and housing the needle after injection.

Still further, according to this invention, a needle hub of a needle can be provided which ensures the safety of a sequence of operations of sucking up fluid through a needle, protecting the needle through which the fluid has been drawn, injecting the fluid into a patient, and housing the needle after injection.

What is claimed is:

1. A needle protector assemblage comprising:
a needle hub (11) of a needle (10);
a protector (32) capable of covering the needle (10); and
a fixing member (31) which is formed as a continuity of the protector (32) and which is fixed to the outer perimeter of the needle hub (11) of the needle (10);
wherein the needle hub (11) comprises:
an outer-perimeter-engaging portion (11b) capable of engaging by friction with the fixing member (31); and
an idling portion (11d) capable of idling the fixing member (31).

2. The needle protector assemblage according to claim 1, wherein one of the outer-perimeter-engaging portion and the idling portion of the needle hub is formed at the near side to the needle and the other at the far side from the needle.

3. The needle protector assemblage according to claim 1, wherein the needle hub further includes a stopper portion for preventing the fixing member from slipping off from the idling portion toward the needle.

4. The needle protector assemblage according to claim 2, wherein the needle hub further includes a stopper portion for preventing the fixing member from slipping off from the idling portion toward the needle.

5. The needle protector assemblage according to claim 1, wherein the inner perimeter of the fixing member includes an engaging portion which engages with the outer perimeter shape of the outer-perimeter-engaging portion of the needle hub.

6. The needle protector assemblage according to claim 2, wherein the inner perimeter of the fixing member includes an engaging portion which engages with the outer perimeter shape of the outer-perimeter-engaging portion of the needle hub.

7. The needle protector assemblage according to claim 3, wherein the inner perimeter of the fixing member includes an engaging portion which engages with the outer perimeter shape of the outer-perimeter-engaging portion of the needle hub.

8. The needle protector assemblage according to claim 4, wherein the inner perimeter of the fixing member includes an engaging portion which engages with the outer perimeter shape of the outer-perimeter-engaging portion of the needle hub.

9. A syringe equipped with the needle protector assemblage according to claim 1, comprising:
a piston which enables the discharge and suction of fluid;
a barrel portion for containing the fluid; and
a tip portion, where the needle protector assemblage is equipped, provided at the tip of the barrel portion.

10. A syringe equipped with the needle protector assemblage according to claim 2, comprising:
a piston which enables the discharge and suction of fluid;
a barrel portion for containing the fluid; and
a tip portion, where the needle protector assemblage is equipped, provided at the tip of the barrel portion.

11. A syringe equipped with the needle protector assemblage according to claim 3, comprising:
a piston which enables the discharge and suction of fluid;
a barrel portion for containing the fluid; and
a tip portion, where the needle protector assemblage is equipped, provided at the tip of the barrel portion.

12. A syringe equipped with the needle protector assemblage according to claim 4, comprising:
a piston which enables the discharge and suction of fluid;
a barrel portion for containing the fluid; and
a tip portion, where the needle protector assemblage is equipped, provided at the tip of the barrel portion.

13. A syringe equipped with the needle protector assemblage according to claim 5, comprising:
a piston which enables the discharge and suction of fluid;
a barrel portion for containing the fluid; and
a tip portion, where the needle protector assemblage is equipped, provided at the tip of the barrel portion.

14. A syringe equipped with the needle protector assemblage according to claim 6, comprising:
a piston which enables the discharge and suction of fluid;
a barrel portion for containing the fluid; and
a tip portion, where the needle protector assemblage is equipped, provided at the tip of the barrel portion.

15. A syringe equipped with the needle protector assemblage according to claim 7, comprising:
a piston which enables the discharge and suction of fluid;
a barrel portion for containing the fluid; and
a tip portion, where the needle protector assemblage is equipped, provided at the tip of the barrel portion.

16. A syringe equipped with the needle protector assemblage according to claim 8, comprising:
a piston which enables the discharge and suction of fluid;
a barrel portion for containing the fluid; and
a tip portion, where the needle protector assemblage is equipped, provided at the tip of the barrel portion.

17. A needle hub comprising:
an outer-perimeter-engaging portion (11b) capable of engaging by friction with a needle protector member (30) which comprises a fixing member (31), which is fixed to the outer perimeter of the needle hub (11) of a needle (10); and
an idling portion (11d) capable of idling the fixing member (31).

18. The needle hub according to claim 17, wherein one of the outer-perimeter-engaging portion and the idling portion of the needle hub is formed at the near side to the needle and the other at the far side from the needle.

19. The needle hub according to claim 17, further including a stopper portion for preventing the fixing member from slipping off from the idling portion toward the needle.

20. The needle hub according to claim 18, further including a stopper portion for preventing the fixing member from slipping off from the idling portion toward the needle.
